(19) United States
(12) Patent Application Publication
(10) Pub. No.: US 2021/0000654 A1
(43) Pub. Date: Jan. 7, 2021

(54) ELECTROMAGNETIC WOUND BANDAGE

(71) Applicant: A.I. Spine LLC, Seattle, WA (US)

(72) Inventors: Omar F. Jimenez, Seattle, WA (US); Yefim I. Safris, Golden Valley, MN (US)

(21) Appl. No.: 16/946,699

(22) Filed: Jul. 1, 2020

Related U.S. Application Data
(60) Provisional application No. 62/869,292, filed on Jul. 1, 2019.

(51) Int. Cl. A61F 13/02

(52) U.S. Cl. CPC A61F 13/0216; A61M 2205/50; A61M 2205/3576; A61M 2205/8262; A61F 2013/00089; A61M 2205/3327; A61M 2205/27; A61M 2205/8287; A61M 2205/3553

(57) ABSTRACT

Disclosed herein are wound bandages that employ electromagnetic elements to accelerate wound healing and therefore reduce the time that such bandages must be worn. In embodiments, a wound bandage can include self-contained electromagnetic circuitry that can be energized to provide low frequency electromagnetic energy to a wound while the bandage is worn over the wound to promote wound healing.

### RELATED APPLICATION

The present application claims the benefit of U.S. Provisional Application No. 62/869,292 filed Jul. 1, 2019, which is incorporated herein in its entirety by reference.

### TECHNICAL FIELD

The present disclosure relates to bandages for covering wounds.

### BACKGROUND

Bandages such as adhesive bandages have been widely utilized for covering scrapes, cuts, incisions and other wounds to enable the wounds to heal while protecting the wounded areas from infection and/or further damage. Adhesive bandages typically include a pad of gauze or other absorbent material for interfacing with the wound positioned on a flexible strip of adhesive material that retains the bandage on the wound. Wounds covered by adhesive bandages can take significant time to heal. This can require frequently needing to remove a bandage and cover the wound with a new bandage as well as cause a prolonged period of time during which a user must wear such a bandage, which can be considered unsightly.

### SUMMARY OF THE INVENTION

Disclosed herein are wound bandages that employ electromagnetic elements to accelerate wound healing and therefore reduce the time that such bandages must be worn. In embodiments, a wound bandage can include self-contained electromagnetic circuitry that can be energized to provide low frequency electromagnetic energy to a wound while the bandage is worn over the wound to promote wound healing.

In an embodiment, an electromagnetic wound bandage includes a flexible wound dressing base configured to conform to a portion of a user's body and an absorbent pad disposed on an underside of the wound dressing base configured to interface with a wound. A flexible electromagnetic base can be disposed on an upper surface of the wound dressing base and configured to flex with the wound dressing base when the wound dressing base is placed on the portion of the user's body. An electromagnetic circuit can disposed on the electromagnetic base and configured to provide a low frequency electromagnetic field to the wound. A flexible cover can be disposed over the electromagnetic circuit to provide a liquid tight barrier to protect the electromagnetic circuit from moisture.

### BRIEF DESCRIPTION OF THE DRAWINGS

Subject matter hereof may be more completely understood in consideration of the following detailed description of various embodiments in connection with the accompanying figures, in which:

FIGS. 1A-1C depict an electromagnetic wound bandage according to the disclosure.

FIGS. 2A-2B depict components of an electromagnetic wound bandage according to the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Unlike high frequency electromagnetic (EM) fields that have been associated with cellular toxicity and carcinogenesis, extremely low frequency (e.g., about 3 to 300 Hz) pulsed EM fields have demonstrated relevant biologic evidence in wound healing. Such extra low frequency EM fields have demonstrated efficiency in DNA repair, improvement in cell metabolism and improvement in cellular oxygenation. The present disclosure leverages this phenomenon by providing a novel bandage having self-contained electromagnetic circuitry capable of generating such low frequency EM fields to accelerate healing of the wound covered by the bandage. This decreases wound healing time in a versatile, portable and low cost manner.

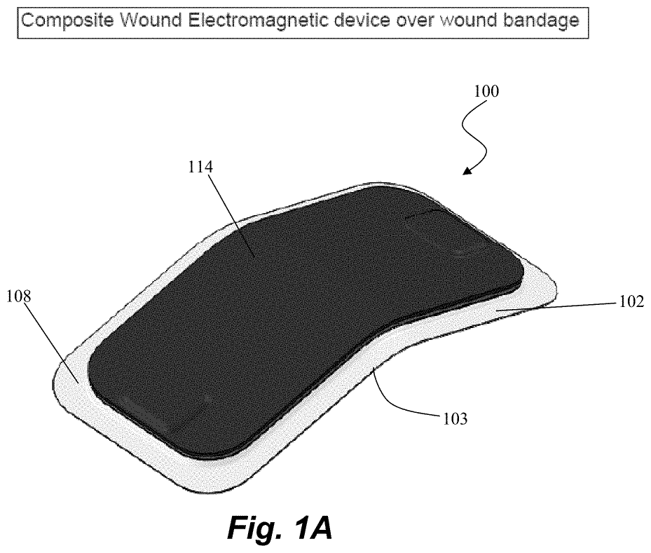

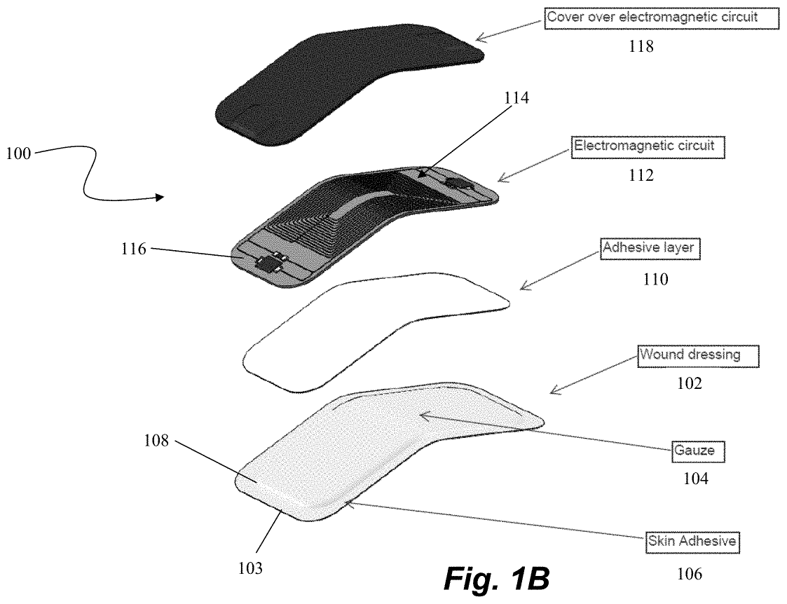

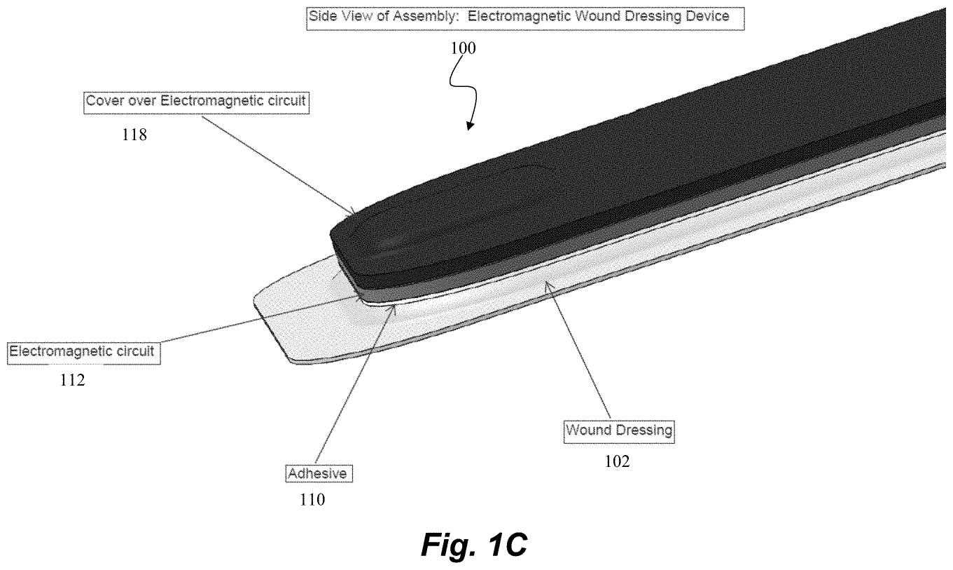

FIGS. 1A-1C depict an electromagnetic wound bandage 100 according to the disclosure. Bandage 100 can include a wound dressing base 102 including an absorbent pad 104 for interfacing with the wound on the underside 103 of wound dressing 102 and an adhesive 106 applied to one or more other portions of the underside 103 of wound dressing 102 to retain the bandage 100 on the user's body. An adhesive layer 110 can be attached to an upper side 108 of wound dressing to enable an electromagnetic (EM) base or breadboard 112 to be attached to wound dressing 102. EM base 112 can include electromagnetic coils 114 disposed on a flexible body 116 that enables EM base 112 to bend and flex along with wound dressing base 102 when being applied to and carried on a wound. A flexible cover 118 can be disposed over EM base 112 to protect the electromagnetic components of EM base 112.

Wound dressing base 102 can comprise a flexible and/or stretchable material to enable the bandage to be applied to and retained on a variety of body areas/parts. In embodiments, base can be made of, for example, a woven fabric material, a plastic material such as, e.g., polyvinylchloride, polyethylene or polyurethane, or a latex material. Wound dressing 102 can therefore be wrapped and/or stretched into a variety of shapes to cover variously located wounds.

The absorbent pad 104 can, for example, be made from a cotton material such as gauze, a hydrogel, or any other absorbent material. The adhesive 106 applied to the underside of wound dressing 102 can be any known adhesive including, e.g., an acrylate. In other embodiments, wound dressing 102 may not have an adhesive 106 and may be retained on the skin by other means, such as, for example, by surgical tape over the top of the wound bandage 100.

Cover 118 can comprise a thin, flexible and watertight material such as a plastic in order to enable cover 118 to flex with the wound bandage 100 when placed on the body and to provide a barrier protecting the EM base circuitry from moisture and other contaminants. Cover 118 may be permanently affixed to EM base 112 or removably attached thereto. Cover 118 may also have a perimeter matching that of flexible body 116 of EM base 112 such that cover 118 is placed over only the top surface of EM base 112 containing EM coils 114 and other circuitry, as shown in FIG. 1C. Alternatively, cover 118 may further include a downwardly extending rim configured to enclose the flexible body 116 of EM base 112 against wound dressing 102.

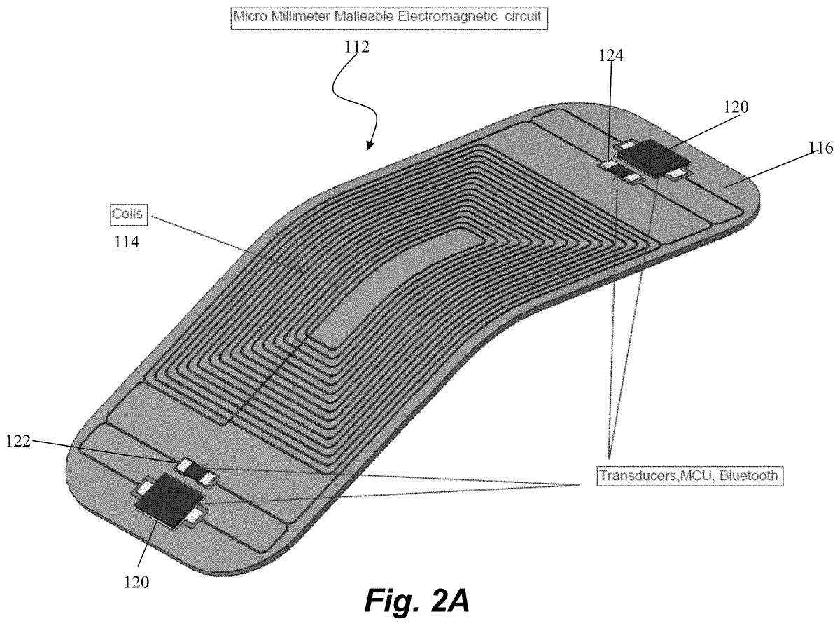

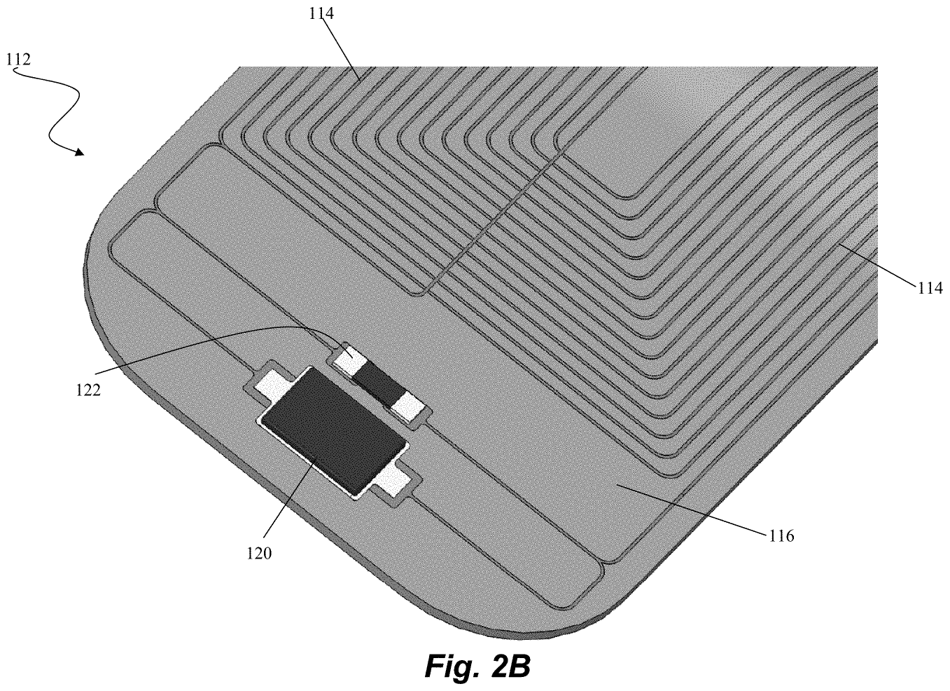

Referring now to FIGS. 2A-2B, further details regarding EM base 112 that includes electrical components to provide the low frequency electromagnetic field to improve wound healing are depicted. In addition to metallic EM coils 114 for transmitting the electromagnetic energy, a pair of ultrasonic transducers 120 that transfer power to the coil 116, a power supply 122 that provides power to the transducers 120 and a MCU (microcontroller) chip 124 that can control device operations including, in embodiments, wireless communications such as by Bluetooth, for example, can be disposed on flexible body 116 of EM base 112. In embodiments, EM coils 114 can be 3d printed onto flexible body 116. In use, the MCU 122 controls the power supply 122 to power the EM coils 144 to generate the electromagnetic field. As noted above, wound healing can be promoted by controlling the system such that an extra low frequency EM field of between about 3 Hz and 300 Hz is generated. The power to run the circuit can come from, for example, an external battery, pulse electromagnetic waves, or a combination thereof controlled by an external remote handheld device via a Bluetooth or other communication link with MCU 124. Remote handheld device can include, for example, a dedicated remote controller designed for use with wound bandage or a smartphone or other remote consumer electronic device having a software application configured to enable communication with MCU. In various embodiments, additional sensors or other components can be included in device to collect data for measuring the healing of the wound such as temperature, impedance and other parameters and such data can be communicated to the external remote device with the MCU.

As noted above, in practice wound bandage 100 can be placed on a wound in a variety of body parts/locations and utilized to provide an EM field to promote wound healing. Wound bandage can be formed in any size or shape and used with any type of wound including surgical incisions involving muscle, fascia, subcutaneous layers and/or epidermis as well as more superficial injuries such as scrapes and small cuts. Wound bandage can also be made small enough to cover, e.g., a finger or facial laceration or any other small skin surface.

Wound bandage 100 can be disposable or reusable. A portion of bandage may be disposable or all of bandage may be disposable. For example, only the wound dressing 102 may be disposable such that the EM base 112 can be detached from the wound dressing 102 and reused with a new wound dressing. The cover 118 may also be disposable or reusable in various embodiments. Alternatively, the entirety of wound bandage may be reusable or disposable.

In another embodiment, EM base 112 can be placed on a breathable film directly on the wound with another breathable cover over the coils to protect the circuit making the device disposable or reusable. In a further embodiment, the EM base 112 is placed in a sterile sealable cover for the purpose of reuse, and, in some embodiments, only the cover would be discarded.

Further details regarding the use of electromagnetic fields in promoting healing in the body can be found in U.S. patent application Ser. No. 16/871,812, which is hereby incorporated by reference in its entirety, and any of the electromagnetic components, configurations and/or settings disclosed therein could be applied to the wound bandages disclosed herein. Various embodiments of systems, devices, and methods have been described herein.

These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions. Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. An electromagnetic wound bandage, comprising:
a flexible wound dressing base configured to conform to a portion of a user's body;
an absorbent pad disposed on an underside of the wound dressing base configured to interface with a wound;
a flexible electromagnetic base disposed on an upper surface of the wound dressing base, the electromagnetic base configured to flex with the wound dressing base when the wound dressing base is placed on the portion of the user's body;
an electromagnetic circuit disposed on the electromagnetic base, the electromagnetic circuit configured to provide a low frequency electromagnetic field to the wound; and
a flexible cover disposed over the electromagnetic circuit to provide a liquid tight barrier to protect the electromagnetic circuit from moisture.

2. The electromagnetic wound bandage of claim 1, wherein the electromagnetic circuit comprises a plurality of metallic coils.

3. The electromagnetic wound bandage of claim 2, wherein the electromagnetic circuit further comprises one or more transducers configured to provide power to the plurality of coils.

4. The electromagnetic wound bandage of claim 3, wherein the electromagnetic circuit further comprises a power supply configured to supply power to the one or more transducers.

5. The electromagnetic wound bandage of claim 4, wherein the power supply is rechargeable with an external device.

6. The electromagnetic wound bandage of claim 1, wherein the electromagnetic circuit comprises a microcontroller configured to control operations of the electromagnetic circuit.

7. The electromagnetic wound bandage of claim 6, wherein the microcontroller is configured to wirelessly communicate with an external device.

8. The electromagnetic wound bandage of claim 7, wherein the external device is a smartphone executing a software application programmed for control of the electromagnetic circuit.

9. The electromagnetic wound bandage of claim 1, wherein the electromagnetic circuit is configured to generate an electromagnetic field between 3 Hz and 300 Hz.

10. The electromagnetic wound bandage of claim 1, further comprising a sensor configured to provide a measure of wound healing.

11. The electromagnetic wound bandage of claim 1, wherein the electromagnetic base is integrally attached to the wound dressing base such that the electromagnetic base is configured to be disposed of along with the wound dressing base.

12. The electromagnetic wound bandage of claim 1, wherein the electromagnetic base is selectively removable from the wound dressing base such that the electromagnetic base is configured to be reused with another wound dressing base after disposal of the wound dressing base.

13. The electromagnetic wound bandage of claim 1, wherein the cover is selectively removable from the electromagnetic base.

14. The electromagnetic wound bandage of claim 1, wherein the cover has an outer perimeter matching an outer perimeter of the electromagnetic base.

15. The electromagnetic wound bandage of claim 1, wherein the cover has a downwardly extending rim configured to surround an outer perimeter of the electromagnetic base.

16. The electromagnetic wound bandage of claim 1, further comprising an adhesive on the underside of the wound dressing base to retain the wound dressing base on the portion of the user's body.
